(19) United States
(12) Patent Application Publication
(10) Pub. No.: US 2026/0069420 A1
(43) Pub. Date: Mar. 12, 2026

(54) Reverse Bolted Greater Trochanter Periprosthetic Claw Plate

(71) Applicant: Baptist Health South Florida, Inc., Coral Gables, FL (US)

(72) Inventor: Alexander van der Ven, Miami, FL (US)

(21) Appl. No.: 19/269,542

(22) Filed: Jul. 15, 2025

Related U.S. Application Data
(60) Provisional application No. 63/671,942, filed on Jul. 16, 2024.

(51) Int. Cl. A61F 2/30; A61F 2/36

(52) U.S. Cl. CPC A61F 2/30739; A61F 2/3607; A61F 2002/30224; A61F 2002/30433; A61F 2002/30514

(57) ABSTRACT

A reverse bolted greater trochanter periprosthetic plate configured to be mounted to a femur of a patient includes a central portion with an inner surface configured to face and/or contact a femoral surface of the femur; a plurality of leg portions extending from the central portion forming a claw configured to grasp a portion of the femur; and a bolt receptor portion extending from the central portion configured for receiving a bolt for securing the plate to the femur during a reverse bolted greater trochanter periprosthetic procedure.

### CROSS REFERENCE TO RELATED APPLICATION

This application claims the benefit of U.S. Provisional Patent Application No. 63/671,942, filed on Jul. 16, 2024, the disclosure of which is hereby incorporated by reference in its entirety.

### BACKGROUND OF THE INVENTION

### Field of the Invention

The present disclosure is generally directed to implantable surgical devices and related surgical techniques and, in particular, to a plate and assembly configured for use in an orthopedic procedure, such as a total hip arthroplasty procedure.

### Description of Related Art

Total hip arthroplasty (THA) is widely identified as one of the most successful interventions in improving function, decreasing pain, and increasing quality of life when performed without complications. See Evans J T, et al., How long does a hip replacement last? A systematic review and meta-analysis of case series and national registry reports with more than 15 years of follow-up, Lancet 393 (10172) February 2019: 647-654.

However, the occurrence of fractures around total hip replacements is often reported as one of the most common causes of morbidity and reoperation for THA procedures. Such fractures have been reported in between 0.5% to 3% of cases. See Bendich, et al., Intraoperative Complications and Early Return to the Operating Room in Total Hip Arthroplasty Performed Through the Direct Anterior and Posterior Approaches, An Institutional Experience of Surgeons After Their Learning Curve, J Arthroplasty (2021) August; 36(8):2829-2835; and Brun O C and Maansson L, Fractures of the greater trochanter following total hip replacement, Hip Int. (2013) March-April; 23(2):143-6.

Of the various fracture types that may occur, fractures of the greater trochanter are considered to be one of the most difficult to manage. Studies have been inconsistent on the need for further management of prosthetic greater trochanteric fracture, but the literature is consistent considering such fractures as being a regularly reported complication. See De Geest, et al., Adverse effects associated with the direct anterior approach for total hip arthroplasty: a Bayesian meta-analysis, Arch Orthop Trauma Surg. (2015) August; 135(8):1183-92.

Fractures can occur at the time of surgery, in the immediate perioperative period, or at a later period of time. Non-displaced fractures potentially can be treated without surgery, but displaced fractures may lead to chronic loss of the abductor unit. There is also a concern that non-displaced fractures may become displaced. Therefore, identification and treatment of such fractures are paramount to the preservation of the abductor function.

Traditional treatment options for fractures occurring at the time of surgery can include wiring techniques, small plates secured by cables, and larger types of lateral plates, which can be fixed with screws and cables. These techniques all require direct visualization of the fracture segment from the lateral perspective, either with a direct lateral incision or a posterolateral incision. See Huang, et al., Fixation options for reconstruction of the greater trochanter in unstable intertrochanteric fracture with arthroplasty, Medicine, 100(26): (Jul. 2, 2021).

Current trends in hip replacement surgery now favor a less invasive and muscle sparing technique, which is referred to as a direct anterior approach (DAA). DAA has been demonstrated to facilitate a quicker recovery, higher outcome scores, and a faster return to work and sport. See Mead, et al., Direct Anterior Approach to Total Hip Arthroplasty Improves the Likelihood of Return to Previous Recreational Activities Compared with Posterior Approach, JAAOS: Global Research and Reviews 6(1):e21.00160, January 2022; and Maldonado, et al., Direct Anterior Approach in Total Hip Arthroplasty Leads to Superior Outcomes at 3-Month Follow-up When Compared With the Posterior Approach: A Matched Study Using Propensity Score Analysis, JAAOS: Global Research and Reviews 3(12):p e19.00118 (December 2019). Current trends now show over 50% of total hip replacements are performed via DAA method for high volume surgeons. Patel, et al., Current Trends in Clinical Practice for the Direct Anterior Approach Total Hip Arthroplasty, J Arthroplasty, 2019 September; 34(9):1987-1993.

However, while benefits of DAA are well known, there is a need for improved surgical devices and tools for THA procedures and/or for surgical techniques that help with the management of complications, such as for treatment of fracture around the greater trochanter. The tools, devices, and methods of the present disclosure are provided to address these issues.

### SUMMARY OF THE INVENTION

According to an aspect of the present disclosure, a reverse bolted greater trochanter periprosthetic plate configured to be mounted to a femur of a patient includes a central portion with an inner surface configured to face and/or contact a femoral surface of the femur; a plurality of leg portions extending from the central portion forming a claw configured to grasp a portion of the femur; and a bolt receptor portion extending from the central portion configured for receiving a bolt for securing the plate to the femur during a reverse bolted greater trochanter periprosthetic procedure.

According to another aspect of the present disclosure, a kit of parts for a surgical procedure includes a plurality of the plates, as previously described, provided in different sizes.

According to another aspect of the present disclosure, an implantable assembly for a surgical procedure includes the reverse bolted greater trochanter periprosthetic plate, as previously described. The implantable assembly also includes a femoral prosthesis with a threaded opening and at least one elongated bolt configured to be inserted through the bolt receptor portion of the plate and the threaded opening of the femoral prosthesis for securing the plate to the prosthesis.

According to another aspect of the disclosure, a surgical method for implanting a femoral prosthesis and the previously described reverse bolted greater trochanter periprosthetic plate for a patient includes initial steps of creating one or more incisions and dissecting and/or retracting fat tissue and muscle to access a surgical site that includes a femur and acetabulum of the patient and hollowing out a medullary canal of the femur. The surgical method also includes steps of placing the femoral prosthesis partially within a hollowed out portion of the medullary canal and inspecting the femoral prosthesis and femur for fractures. When a fracture(s) is identified in a greater trochanter of the femur, the method also includes a step of implanting the reverse bolted greater trochanter periprosthetic plate around the greater trochanter and fixed to the femoral prosthesis to stabilize and support the identified fracture(s).

Non-limiting illustrative examples of embodiments of the present disclosure will now be described in the following numbered clauses.
Clause 1: A reverse bolted greater trochanter periprosthetic plate configured to be mounted to a femur of a patient, the plate comprising: a central portion comprising an inner surface configured to face and/or contact a femoral surface of the femur; a plurality of leg portions extending from the central portion forming a claw configured to grasp a portion of the femur; and a bolt receptor portion extending from the central portion configured for receiving a bolt for securing the plate to the femur during a reverse bolted greater trochanter periprosthetic procedure.
Clause 2: The plate of clause 1, wherein the femoral surface comprises a surface of a greater trochanter of the femur.
Clause 3: The plate of clause 1 or clause 2, wherein the femoral surface comprises a medial surface of a greater trochanter of the femur.
Clause 4: The plate of any of clauses 1-3, wherein the bolt receptor portion comprises a threaded annular opening configured to receive a threaded bolt.
Clause 5: The plate of any of clauses 1-4, wherein the bolt receptor portion extends from a top of the central portion.
Clause 6: The plate of clause 5, wherein the plurality of leg portions extend from a bottom and sides of the central portion.
Clause 7: The plate of any of clauses 1-6, wherein the plurality of leg portions each comprise an intermediate portion that is angled relative to the central portion and a distal portion that is angled relative to the intermediate portion.
Clause 8: The plate of clause 7, wherein the intermediate portion is angled relative to the central portion by from about 10 degrees to 50 degrees, and wherein the distal portion is angled relative to the intermediate portion by about 90 degrees to about 160 degrees.
Clause 9: The plate of any of clauses 1-8, wherein the plurality of leg portions are configured to provide fixation for the greater trochanter of the patient in a superior direction and in at least one additional direction.
Clause 10: The plate of clause 9, wherein the at least one additional direction comprises an anterior direction or a posterior direction.
Clause 11: The plate of any of clauses 1-10, wherein the plurality of leg portions comprises four leg portions extending from a bottom half of the central portion.
Clause 12: The plate of any of clauses 1-11, comprising at least one of a titanium alloy, cobalt chromium, or stainless steel.
Clause 13: A kit of parts for a surgical procedure, comprising: a plurality of the plates of clause 1 provided in different sizes.
Clause 14: The kit of clause 13, further comprising a plurality of elongated bolts of different sizes configured for use with different sized cylindrical spacers.
Clause 15: The kit of clause 13 or clause 14, wherein the plurality of plates are provided in sizes of small, medium, and large, and/or configured for use with one or both of a right hip and a left hip.
Clause 16: The kit of any of clauses 13-15, further comprising at least one spacer configured to be connected between the plate and a femoral prosthesis for increasing a distance between the plate and an outer surface of the femur.
Clause 17: The kit of clause 14, further comprising a spring washer configured to be connected to the plurality of elongated bolts for preventing backout.
Clause 18: The kit of clause 17, wherein the spring washer is integral with one of the plurality of elongated bolts.
Clause 19: The kit of any of clauses 13-18, further comprising a femoral prosthesis comprising a trunnion, a lateral shoulder, and a stem.
Clause 20: An implantable assembly, comprising: the reverse bolted greater trochanter periprosthetic plate of any of clauses 1-12; a femoral prosthesis comprising a threaded opening; and at least one elongated bolt configured to be inserted through the bolt receptor portion of the plate and the threaded opening of the femoral prosthesis for securing the plate to the femoral prosthesis.
Clause 21: The assembly of clause 20, further comprising one or more spacers for adjusting an elevation of the plate relative to the femoral surface to accommodate size differences between patients.
Clause 22: The assembly of clause 20 or clause 21, further comprising a spring washer configured to prevent the at least one elongated bolt from backing out from the threaded opening of the femoral prosthesis.
Clause 23: The assembly of any of clauses 20-22, wherein the femoral prosthesis comprises a trunnion, a lateral shoulder extending distally from the trunnion, and a stem extending distally from the lateral shoulder.
Clause 24: The assembly of clause 23, wherein the threaded opening extends through the lateral shoulder of the femoral prosthesis.
Clause 25: The assembly of clause 23 or clause 24, wherein the stem of the femoral prosthesis comprises a porous femoral stem configured to be implanted by a cementless technique.
Clause 26: A surgical method for implanting a femoral prosthesis and the reverse bolted greater trochanter periprosthetic plate of any of clauses 1-12 for a patient, the method comprising: creating one or more incisions and dissecting and/or retracting fat tissue and muscle to access a surgical site comprising a femur and acetabulum of the patient; hollowing out a medullary canal of the femur; placing the femoral prosthesis partially within a hollowed out portion of the medullary canal; inspecting the femoral prosthesis and femur for fractures; and when a fracture(s) is identified in a greater trochanter of the femur, implanting the reverse bolted greater trochanter periprosthetic plate around the greater trochanter and fixed to the femoral prosthesis to stabilize and support the identified fracture(s).
Clause 27: The surgical method of clause 26, wherein implanting the reverse bolted greater trochanter periprosthetic plate comprises: clearing a lateral shoulder of the femoral prosthesis of tissue to provide a location for fixation of the plate to the lateral shoulder of the femur; placing one or more of the leg portions of the plate through muscle tendons and around the greater trochanter bone; bringing the bolt receptor of the plate close to a threaded recess of the femoral prosthesis; and placing a screw, threaded bolt, or fastener through the bolt receptor of the plate and into the threaded recess of the femoral prosthesis, thereby fixing the plate and the femoral prosthesis.
Clause 28: The surgical method of clause 27, wherein implanting the reverse bolted greater trochanter periprosthetic plate further comprises positioning a cylindrical spacer(s) between the bolt receptor of the plate and the recess opening of the femoral prosthesis to accommodate a size and shape of the greater trochanter.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limit of the invention.

FIG. 1A is a perspective view of a femoral prosthesis for a THA procedure;

FIG. 1B is a perspective view of the femoral prosthesis of FIG. 1A with a screw inserted through the threaded opening of the prosthesis;

FIGS. 2A and 2B are perspective views showing the femoral prosthesis of FIG. 1A secured to a femur with a displaced fracture of the greater trochanter;

FIGS. 3A-3C are perspective views of a reverse bolted greater trochanter periprosthetic plate, according to aspects of the present disclosure;

FIG. 3D shows an assembly including the plate of FIGS. 3A-3C, along with screws and a lengthening shaft, for securing the plate to a patient's femur, according to an aspect of the present disclosure; and

FIGS. 4A-4C are perspective views showing the plate of FIGS. 3A-3C secured to the femoral prosthesis and securing a fracture of the greater trochanter according to surgical techniques of the present disclosure.

### DESCRIPTION OF THE INVENTION

As used herein, the singular form of “a”, “an”, and “the” include plural referents unless the context clearly states otherwise.

As used herein, the terms “right”, “left”, “top”, “bottom”, and derivatives thereof shall relate to the invention as it is oriented in the drawing figures. As used herein, the term “proximal” refers to a structure that is closer to a central portion, center of mass, or trunk of a body of a patient. By contrast, the term “distal” refers to a structure that is closer to a peripheral portion of the body and spaced apart from the center of mass or central portion of the body. For example, the thoracic cavity is proximal relative to the arms, hands, fingers, or other peripheral digits of the patient. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

For the purposes of this specification, unless otherwise indicated, all numbers expressing, for example, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention. Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “comprising,” “comprise” or “comprised,” and variations thereof, are meant to be open ended. As used herein, the term “patient” or “subject” refers to members of the animal kingdom including but not limited to human beings.

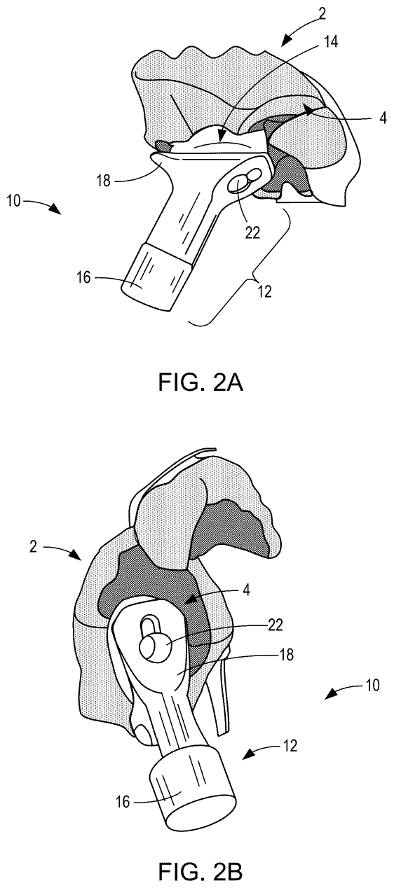

The present disclosure is directed to surgical devices, tools, and methods for use in a total hip arthroplasty (THA) procedure, in which an implantable device, such as femoral prosthesis 10, is directly secured to a femur 2 (shown in FIGS. 2A and 2B) of a patient and a separate plate 110 is connected to the femoral prosthesis 10 directly for supporting a femoral fracture 4 (shown in FIGS. 2A and 2B), such as a fracture 4 of the greater trochanter. In some examples, the plate 110 can also be adopted for use in other surgical procedures, such as surgical procedures for the shoulder or knee.

The plate 110, which is referred to herein as a reverse bolted greater trochanter periprosthetic plate 110, can be configured to secure either a segment or an entirety of the greater trochanter in order to stabilize and promote healing of a fractured region of the greater trochanter. As used herein, the “patient” can be any species of the human or animal kingdom having a pelvis and femur joined together at a hip joint. Non-limiting examples of patients include mammal(s), such as human(s) and/or non-mammalian animal(s). Non-limiting examples of mammal(s) include primate(s) and/or non-primate(s). Primate(s) include human(s) and non-human primate(s), including but not limited to male(s), female(s), adult(s) and children. Non-limiting examples of non-human primate(s) include monkey(s) and/or ape(s), for example chimpanzee(s). Non-limiting examples of non-primate(s) include cattle (such as cow(s), bull(s) and/or calves), pig(s), camel(s), llama(s), alpaca(s), horse(s), donkey(s), goat(s), rabbit(s), sheep, hamster(s), guinea pig(s), cat(s), dog(s), rat(s), mice, lion(s), whale(s), and/or dolphin(s). Non-limiting examples of non-mammalian animal(s) include bird(s) (e.g., duck(s) or geese), reptile(s) (e.g., lizard(s), snake(s), or alligator(s)), amphibian(s) (e.g., frog(s)), and/or fish. In some examples, the animals can be zoological animals, human pets and/or wild animals.

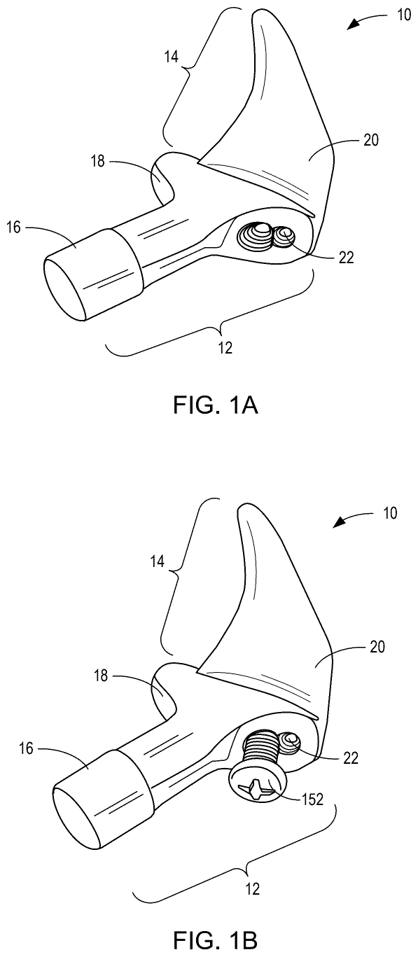

An exemplary femoral prosthesis 10, which can be used for the total hip arthroplasty procedures of the present disclosure, is shown in FIGS. 1A-2B. Femoral prosthesis devices which can be used with the surgical techniques disclosed herein are made by many manufacturers including, for example, Depuy (Johnson & Johnson), Zimmer/Biomet, Stryker, and others. As shown in FIGS. 1A-2B, in some examples, the femoral prosthesis 10 comprises a proximal portion 12, which can comprise a neck, a lateral shoulder 18, and trunnion 16. The trunnion 16 is configured to be connected or fixed to a modular articular prosthetic femoral head (not shown) forming the replacement hip joint. Upon implantation, the prosthetic femoral head (not shown) is configured to articulate with a pelvis or acetabular component.

The femoral prosthesis 10 can further comprise a distal portion 14 comprising a collar, blade, and/or femoral stem 20, which can be connected to or integral with and extends distally from the proximal portion 12. The distal portion 14 comprising the femoral stem 20 can be configured to be connected to the femur 2 by, for example, inserting the stem 20 into a hollow cavity of the femur 2. The femoral stem 20 can be cemented, meaning that a bone cement (e.g., polymethylmethacrylate (PMMA) bone cement) is used to fix a surface of the stem 20 to bone within the hollow cavity of the femur 2. In other examples, the interface between the femoral stem 20 and bone can be cementless and configured to permit natural bone to grow into the stem 20, thereby fixing the stem 20 to the femur 2.

In some examples, the proximal portion 12 and/or the distal portion 14 of the femoral prosthesis 10 can be formed from a biocompatible metal material, such as titanium alloy (e.g., Ti64 alloy), cobalt chromium alloy, and/or stainless steel. In some instances, the distal portion 14 of the prosthesis 10 can be porous, as discussed above, allowing the natural bone to grow on and into the porous surface for fixing the stem 20 to bone without using bone cement.

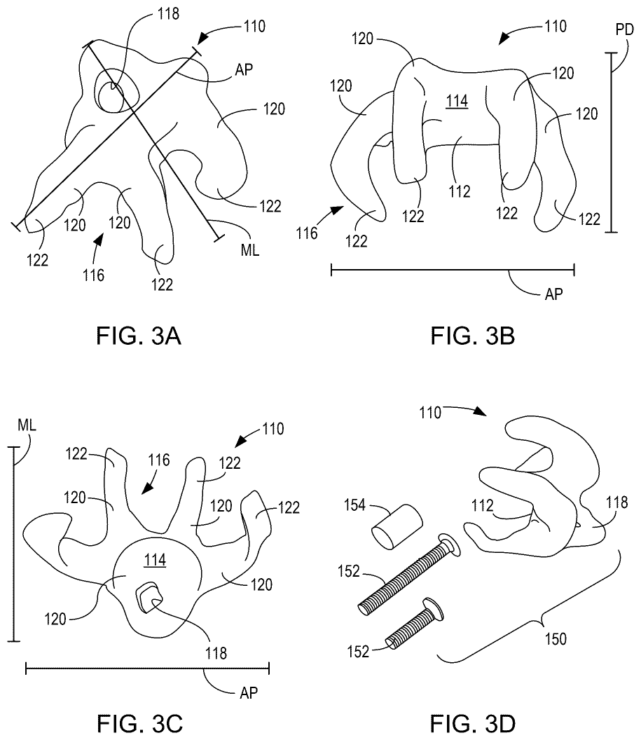

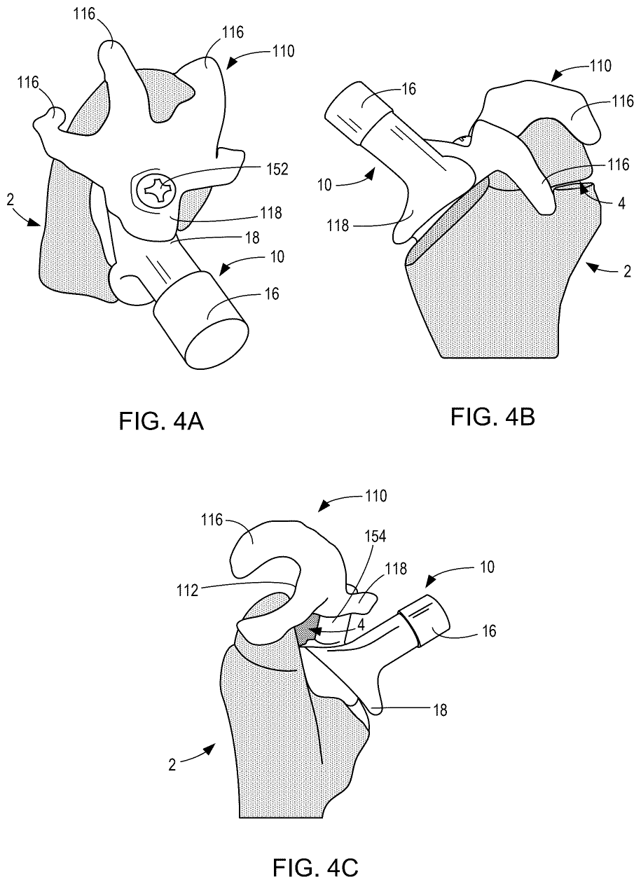

As shown in FIGS. 1A and 1B, the femoral prosthesis 10 further comprises a threaded recess or opening 22 in a top portion of the lateral shoulder 18. The threaded recess or opening 22 can be an enclosed hole including an open top end on an outer surface of the prosthesis 10 and a closed bottom. In other examples, the opening 22 can be a through-hole extending through, for example, the lateral shoulder 18 of the prosthesis 10. In conventional hip replacement procedures, the threaded recess or opening 22 is used solely for extraction of the prosthesis 10 if ever required. For example, the opening 22 can be configured to receive a removal tool sized for insertion into and configured to engage the threaded opening 22. In conventional procedures, the threaded recess or opening 22 generally does not perform another function. However, as described in further detail in connection with FIGS. 3A-4C, in the present disclosure, the threaded opening 22 is used for securing the plate 110 to the femoral prosthesis 10. The plate 110 and prosthesis 10 can be secured to the greater trochanter and femur 2 by the surgical methods disclosed herein.

The present disclosure is also directed to the novel surgical method for a total hip arthroplasty procedure that includes attaching the plate 110 to the femoral prosthesis 10 in order to stabilize a trochanter segment with a fracture. In arriving at the novel surgical method, the present inventor considered that success of total hip arthroplasty is often based on maintaining the abductor lever, which can be critical to the efficient walking cycle in upright humans. In addition, the greater trochanter comprises the anchor point for the gluteus medius and gluteus minimus muscles, which are the most important muscles that affect upright walking and balance. Any uncorrected fracture of the greater trochanter may affect the ability of the gluteus medius and gluteus minimus muscles to offer sufficient support during walking and other exercises. Also, continuity of the muscle tendon to bone interface can be a critical unit around the hip. It has been determined that loss of this functioning unit, which may occur when there are fractures in the greater trochanter, leads to chronic pain, disability, limp, prosthetic hip dislocations, dissatisfaction, and need for further surgical interventions. See Masonis et al., Surgical Approach, Abductor Function, and Total Hip Arthroplasty Dislocation, Clinical Orthopaedics and Related Research 405 (2002), p 46-53, December 2002. Therefore, the present inventor determined that an implantable assembly configured to be connected to the greater trochanter and to support the greater trochanter to allow fractures to heal may provide superior results and/or improved outcomes compared to other surgical devices and techniques.

The present inventor has also recognized that one concern with the minimally invasive DAA incision is dealing with and visualizing greater trochanteric fractures. As opposed to lateral exposures, which gives direct access to the fracture segment, the fracture is visualized on the inside from the medial perspective with the DAA procedure. Furthermore, there are currently no described techniques for fixation of the greater trochanter from a DAA unless a separate incision is made.

To address such concerns, the present invention uses a novel greater trochanteric fixation technique from a medial perspective, or inside out view, for fixation of the greater trochanter. In other examples, the fixation technique can be performed from a medial perspective in a direct anterior approach, as well as from a medial perspective from a direct posterior, posterolateral, direct lateral, anterolateral, or superior approach. In particular, as described in further detail herein, the surgical technique of the present invention involves attaching the femoral prosthesis 10 to the plate 110 which, in turn, is pressed against or connected to the greater trochanter to reduce and secure a fractured segment. As described herein, this fixation technique addresses the lack of options available for trochanteric fixation from a DAA. Specifically, the fixation techniques disclosed herein provide a novel opportunity for fixation of a trochanteric plate 110 to the actual femoral prosthesis 10, rather than the unpredictable distal bone, as occurs in DAA procedures and as shown in FIGS. 2A and 2B. In examples, the fixation technique disclosed herein can also be used for any other approaches for total hip arthroplasty.

### Reverse Bolted Greater Trochanter Periprosthetic Plate

FIGS. 3A-3D show an example of a reverse bolted greater trochanter periprosthetic plate 110, which can be used with the surgical techniques of the present disclosure. The plate 110 can be configured to be secured to and/or in contact with a medial, lateral, anterior, posterior or superior-portion of the greater trochanter. Benefits of the plates 110 of the present disclosure can include: low implementation costs because it is likely that trials will not be needed in order to obtain approval for the plate 110; an ability to be used with implants (e.g., femoral prosthesis devices 10) manufactured by a variety of manufacturers (e.g., Depuy/Johnson & Johnson, Stryker, Zimmer/Biomet, Smith & Nephew, and others); that surgical techniques using the plate 110 will require only minimal surgeon training; and that the plate 110 and femoral prosthesis 10 can be load bearing meaning that patients can walk on the implants immediately.

As previously described, the plate 110 is configured to be mounted or secured to a femur 2 of a patient. The plate 110 can be formed from various biocompatible materials commonly used for medical implants including titanium alloy (e.g., Ti64 alloy), cobalt chromium alloy, or stainless steel. Generally, mixing metals in medical implants is to be avoided. Therefore, often the plate 110 and femoral prosthesis 10 are formed from the same type of metal material.

Dimensions of the plate 110 can be selected based on typical anatomical measurements for an average patient and, in particular, for typical dimensions of the greater trochanter. In some examples, the plate 110 has the following dimensions: medial to lateral (ML) length on a coronal view of about 35 mm to 55 mm; an anterior to posterior (AP) length on an axial view of about 50 mm to about 75 mm; and a proximal to distal (PD) dimension on a sagittal view of about 25 mm to about 45 mm. In some examples, plates 110 can be provided in a variety of sizes (e.g., small, medium, and large) in order to facilitate treatment for a wider variety of patient sizes. For example, a small plate 110 may have the following dimensions: about 36 mm ML, about 52 mm AP, and about 28 mm PD. Dimensions for a medium plate may be about 41 mm ML, about 62 mm AP, and about 35 mm PD. Dimensions for a large plage 110 may be about 50 mm ML, about 74 mm AP, and about 42 mm PD. Furthermore, as described in detail hereinafter, spacers and similar fasteners can be used to accommodate patients with greater trochanters of different shapes and sizes.

In some examples, the greater trochanter periprosthetic plate 110 comprises a central portion 112 comprising an inner surface 114 configured to face and/or to be pressed against a femoral surface of the femur 2. For example, the central portion 112 can comprise an inwardly facing surface 114 with a curvature configured to match or correspond with a curvature of an exterior surface of at least a portion of the greater trochanter of the femur 2. The plate 110 is generally claw-shaped having multiple leg portions 116 extending from the central portion 112 forming a claw configured to grasp and/or retain a portion of the femur 2, such as a portion of the greater trochanter. For example, the plate 114 can include two, three, four, six, eight, or more leg portions 116 positioned to grasp and/or surround the greater trochanter.

In some examples, the plate 110 is configured to grasp or retain a fractured portion 4 of the greater trochanter restricting movement of a fractured portion 4 of bone in at least two directions. For example, the plate 110 can include one or more leg portions 116 configured to support the fracture 4 restricting movement in a superior direction. As shown in FIGS. 3A-3D, the plate 110 includes two center leg portions 116 that restrict the movement in this superior direction. The plate 110 shown in FIGS. 3A-3D also includes outer leg portions 116 that restrict or prevent movement of a fractured portion 4 of the femur 2 in either an anterior direction or a posterior direction. Generally, the plate 110 need only restrict movement in one of the anterior direction or the posterior direction depending upon whether the plate 110 is being used for a right hip replacement procedure or a left hip replacement procedure. The plate 110 shown in FIGS. 3A-3D is a universal plate including both an outer leg portion 116 for restricting the anterior movement of the fractured portion 4 of the femur 2 and an opposing outer leg portion 116 for restricting posterior movement of the fractured portion 4 of the femur 2. However, since both anterior and posterior leg portions 116 generally are not needed, optionally, a surgeon may remove one of the outer leg portions 116 of the plate 110 prior to implantation, depending upon which direction needs to be supported (i.e., whether the hip replacement is for a left hip or a right hip).

The plate 110 further comprises a threaded annular opening or bolt receptor 118 extending from the central portion 112 configured for receiving a bolt for securing the plate 110 to the femoral prosthesis 10 during a reverse bolted greater trochanter periprosthetic procedure. In some examples, the bolt receptor 118 or threaded annular opening is configured to receive a threaded bolt or screw. As shown in FIGS. 4A-4C, the bolt receptor 118 can be positioned to align with the threaded opening 22 of the femoral prosthesis 10 so that the threaded bolt or screw passes through the bolt receptor 118 of the plate 110 and into the threaded recess or opening 22, thereby securing the plate 110 to the femoral prosthesis 10. Beneficially, the plate 110 can be used with different types of femoral prosthesis 10 by different manufacturers, which all tend to include a threaded recess or opening 22 at a similar location of the femoral component. In particular, the plate 110 can be used with any of a variety of femoral implants or prosthesis 10, provided that the device includes a threaded opening 22 or recess positioned on or near the lateral shoulder 18. It is noted that while not all hip stems have threaded recesses or holes, most stems do include some type of opening or recess for removal. If the opening or recess of the implant or prosthesis being used is not threaded, then a mechanical latch or similar mechanical fastener can be used for securing the plate 110 to the unthreaded recess or opening.

As shown in FIGS. 3A-3C, the leg portions 116 of the plate 110 extend radially outward from the central portion 112. Positioning and dimensions of the leg portions 116 can be selected based on average anatomical measurements for the greater trochanter. For example, the leg portions 116 can have a total length of about 25 mm to about 50 mm or, preferably about 28 mm to about 44 mm. Also, as previously described, multiple plates 110 of different sizes (e.g., small, medium, and large) can be provided allowing the surgeon to select an appropriate size for a particular patient.

In some examples, as shown in FIGS. 3A-3C, the plate 110 includes four leg portions 116 extending from a bottom and sides of the central portion 112. The bolt receptor 118 extends from the top of the central portion 112. In some examples, the leg portions 116 can be finger-shaped including axially connected portions or segments. For example, the leg portions 116 can include an intermediate portion or segment 120 that is angled relative to the central portion 112 and a distal portion or segment 122 axially connected to the intermediate segment 120 that is angled relative to the intermediate segment 120. In examples, the intermediate segment 120 can be angled relative to the central portion by from about 10 degrees to 50 degrees. The distal segment 122 can be angled relative to the intermediate portion by about 90 degrees to about 160 degrees.

### Surgical Kit and Femoral Replacement Assembly

As previously described, the plate 110 is intended to be a component of a femoral replacement assembly 150. As shown in FIGS. 3D and 4A-4C, the assembly 150 can include the plate 110 and an elongated fastener 152, such as a threaded bolt, for securing the plate 110 to a threaded opening 22 of a femoral prosthesis 10. Generally, the femoral prosthesis 10 is provided separately from the plate 110 and fastener 152. For example, the femoral prosthesis 10 can be provided by a total hip manufacturer, along with other components needed for total hip replacement surgery. The plate 110 and elongated fastener 152 can be provided or used only when a fracture 4 is identified during or after surgery. In examples, the elongated fastener 152 can be a screw (e.g., a screw comprising a hexagonal or torx screw head), lengthening shaft, or another threaded fastener, such as a bracket or threaded rod.

The assembly 150 can also include one or more lengthening shafts or spacers 154 for adjusting an elevation of the plate 110 relative to the femoral surface and/or to the femoral prosthesis 10 to accommodate size differences between patients or variations in the depth of the femoral component shoulder in relation to the native bone. For example, as shown most clearly in FIG. 4C, the spacer 154 can be inserted over the threaded opening 22 of the femoral prosthesis 10 in order to separate the central portion 112 of the plate 110 from the lateral shoulder 18 of the femoral prosthesis 10. In some examples, a kit may be provided with brackets or screws of different lengths to be used with different spacers.

In some examples, the assembly 150 can also include a spring washer (not shown) configured to prevent the elongated fastener 152 from backing out from the threaded opening 22 of the femoral prosthesis 10. In particular, as most of the threaded recess openings 22 on the femoral prosthesis 10 are cylindrical, the spring washer may be needed to secure the elongated fastener 152 to the prosthesis 10 preventing back out. In some examples, the spring washer can be threaded and configured to engage corresponding threads at a distal end of the elongated fastener 152 for holding the fastener 152 in place. In other examples, the spring washer can be integral with the elongated fastener 152 and/or mounted to the fastener 152 in another manner.

In some examples, the assembly 150 can be provided to the surgeon as a kit of parts including the components needed for performing the reverse bolted greater trochanter surgical technique. In some examples, the kit can be provided as a single-use pack, meaning that any unused components can be discarded after the surgical procedure is complete. For example, the kit can comprise multiple plates 110 of different sizes, such as the small, medium, and large plates 110, as previously described. Alternatively, plates 110 can be provided as three different sized kits (i.e. a kit with a small plate 110, a kit with a medium plate 110, and a kit with a large plate 110). Each kit can also comprise screws or fasteners 152 sized for the particular plate 110 and femoral prosthesis 10 provided in the kit.

The kit also includes the one or more elongated fasteners 152, such as the threaded bolts or screws as previously described, for securing the plate 110 to the femoral prosthesis 10. The fasteners 152 can be proved in different lengths to accommodate size differences between patients. In addition, the kit can include the one or more spacers 154, as previously described, configured to be connected between the femoral prosthesis 10 and plate 110 for increasing spacing between the components. The kit can also include the spring washer configured to be connected to the fastener 152 for preventing the fastener 152 from backing out of the threaded opening 22 of the femoral prosthesis 10 and/or bolt receptor 118 of the plate 110.

While uncommon, in some examples, the kit can also include the femoral prosthesis 10 itself which, as previously described, comprises the trunnion 16, lateral shoulder 18, and femoral stem 20 or blade. The femoral prosthesis 10 can be a commercially available implantable device, as are currently used for many THA procedures. Exemplary femoral prosthesis 10 are available from many manufacturers including Depuy (Johnson & Johnson), Zimmer/Biomet, Stryker, and others.

### Reverse Bolted Greater Trochanter Periprosthetic Plate Surgical Technique

As previously described, the plate 110 disclosed herein is configured for use in a reverse bolted greater trochanter periprosthetic surgical technique. The plate 110 can also be used in connection with other described approaches for total hip arthroplasty. In other examples, as previously described, the plate 110 can be adapted for use for knee or shoulder surgery.

In some examples, the surgical technique for implanting a femoral prosthesis device 10 and plate 110 includes the following steps. First, a surgeon makes a surgical incision in a proper location to approach the hip according to the surgeon's preference and indications. The surgeon may then dissect fat tissue and retract muscles in order to approach the surgical site. Once the femur 2, acetabulum, or previous components are accessible, the surgeon can perform the indicated surgery, whether a partial hip replacement, total hip replacement, or hip replacement revision. If there is no preexisting prosthetic hip, a femoral neck osteotomy is usually made at the neck of the femur 2. The acetabulum is then inspected, prepared with a reamer, and an acetabular component is placed with an appropriate bearing. The femur 2 is then prepared by hollowing out the medullary canal. A trial or permanent prosthesis (e.g., the femoral prosthesis 10 shown in FIGS. 1A-2B and 4A-4C) is then placed with a trial or permanent articulating femoral head component.

Once the femoral prosthesis 10 is in place, the surgeon inspects the femoral prosthesis 10 and femur 2, including the greater trochanter, for failure or fractures. If fractures are identified in the greater trochanter, the surgeon can use the reverse bolted greater trochanter periprosthetic plate 110, as described herein, in order to stabilize and support the identified fracture 4. Steps for implanting the plate 110 can include clearing a lateral shoulder 18 of the femoral prosthesis 10 of tissue to provide a clear path for fixation of the plate 110 to lateral shoulder 18 and the placing leg portion(s) 116 of the plate 110 through muscle tendons (e.g., tendons of the gluteus medius) and around the greater trochanter bone. Next, the surgeon can bring the opening or bolt receptor 118 of the plate 110 close to the threaded recess 22 of the femoral prosthesis 10. The surgeon then measures to see if a cylindrical spacer(s) 154 is needed to accommodate the size and shape of the greater trochanter. The surgeon then places a screw, threaded bolt, or fastener 152 through the opening or bolt receptor 118 of the plate 110 as well as through any required spaces 154, and into the recess or opening 22 of the femoral prosthesis 10. The screw or fastener 152 can then be tightened with a torque limiting wrench to ensure secure fixation between the plate 110 and femoral prosthesis 10. Once the plate 110 and femoral prosthesis 10 are in place, the surgeon confirms that the fractured trochanteric segment is properly positioned and, once positioning is confirmed, can close the wound in a standard fashion.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. Furthermore, it is to be understood that the present invention contemplates that, to the extent possible, one or more features of any embodiment can be combined with one or more features of any other embodiment.

The invention claimed is:

1. A reverse bolted greater trochanter periprosthetic plate configured to be mounted to a femur of a patient, the plate comprising:
a central portion comprising an inner surface configured to face and/or contact a femoral surface of the femur;
a plurality of leg portions extending from the central portion forming a claw configured to grasp a portion of the femur; and
a bolt receptor portion extending from the central portion configured for receiving a bolt for securing the plate to the femur during a reverse bolted greater trochanter periprosthetic procedure.

2. The plate of claim 1, wherein the femoral surface comprises a surface of a greater trochanter of the femur.

3. The plate of claim 1, wherein the femoral surface comprises a medial surface of a greater trochanter of the femur 4. The plate of claim 1, wherein the bolt receptor portion comprises a threaded annular opening configured to receive a threaded bolt.

5. The plate of claim 1, wherein the bolt receptor portion extends from a top of the central portion.

6. The plate of claim 5, wherein the plurality of leg portions extend from a bottom and sides of the central portion.

7. The plate of claim 1, wherein the plurality of leg portions each comprise an intermediate portion that is angled relative to the central portion and a distal portion that is angled relative to the intermediate portion.

8. The plate of claim 7, wherein the intermediate portion is angled relative to the central portion by from about 10 degrees to 50 degrees, and wherein the distal portion is angled relative to the intermediate portion by about 90 degrees to about 160 degrees.

9. The plate of claim 1, wherein the plurality of leg portions are configured to provide fixation for the greater trochanter of the patient in a superior direction and in at least one additional direction.

10. The plate of claim 9, wherein the at least one additional direction comprises an anterior direction or a posterior direction.

11. The plate of claim 1, wherein the plurality of leg portions comprises four leg portions extending from a bottom half of the central portion.

12. The plate of claim 1, comprising at least one of a titanium alloy, cobalt chromium, or stainless steel.

13. A kit of parts for a surgical procedure, comprising:
a plurality of the plates of claim 1 provided in different sizes.

14. The kit of claim 13, further comprising a plurality of elongated bolts of different sizes configured for use with different sized cylindrical spacers.

15. The kit of claim 13, wherein the plurality of plates are provided in sizes of small, medium, and large, and configured for use with both a right hip and a left hip.

16. An implantable assembly, comprising:
the reverse bolted greater trochanter periprosthetic plate of claim 1;
a femoral prosthesis comprising a threaded opening; and
at least one elongated bolt configured to be inserted through the bolt receptor portion of the plate and the threaded opening of the femoral prosthesis for securing the plate to the femoral prosthesis.

17. The assembly of claim 16, further comprising one or more spacers for adjusting an elevation of the plate relative to the femoral surface to accommodate size differences between patients.

18. The assembly of claim 17, further comprising a spring washer configured to prevent the at least one elongated bolt from backing out from the threaded opening of the femoral prosthesis.

19. A surgical method for implanting a femoral prosthesis and the reverse bolted greater trochanter periprosthetic plate of claim 1 for a patient, the method comprising:
creating one or more incisions and dissecting and/or retracting fat tissue and muscle to access a surgical site comprising a femur and acetabulum of the patient;
hollowing out a medullary canal of the femur;
placing the femoral prosthesis partially within a hollowed out portion of the medullary canal;
inspecting the femoral prosthesis and femur for fractures; and
when a fracture(s) is identified in a greater trochanter of the femur, implanting the reverse bolted greater trochanter periprosthetic plate around the greater trochanter and fixed to the femoral prosthesis to stabilize and support the identified fracture(s).

20. The surgical method of claim 19, wherein implanting the reverse bolted greater trochanter periprosthetic plate comprises:
clearing a lateral shoulder of the femoral prosthesis of tissue to provide a location for fixation of the plate to the lateral shoulder of the femur;
placing one or more of the leg portions of the plate through muscle tendons and around the greater trochanter bone;
bringing the bolt receptor of the plate close to a threaded recess of the femoral prosthesis; and
placing a screw, threaded bolt, or fastener through the bolt receptor of the plate and into the threaded recess of the femoral prosthesis, thereby fixing the plate and the femoral prosthesis.
